(19) United States
(12) Patent Application Publication
(10) Pub. No.: US 2023/0046136 A1
(43) Pub. Date: Feb. 16, 2023

(54) FLEXIBLE LID FOR FERMENTATION CONTAINER

(71) Applicants: Gerald Ward, II, St. Petersburg, FL (US); Ruth Edwards, St. Petersburg, FL (US)

(72) Inventors: Gerald Ward, II, St. Petersburg, FL (US); Ruth Edwards, St. Petersburg, FL (US)

(21) Appl. No.: 17/400,314

(22) Filed: Aug. 12, 2021

(51) Int. Cl. C12M 1/00; A47J 31/44

(52) U.S. Cl. CPC C12M 23/38; C12M 23/26; A47J 31/4407

(57) ABSTRACT

A flexible lid may include a body including an upper portion connected approximately perpendicular to a cylindrical sidewall, the body defining a recess and inner wall constructed and arranged to facilitate the attachment of the flexible lid to a container. The flexible lid may include a plurality of ribs integrally formed with the cylindrical sidewall constructed and arranged to facilitate the elastic attachment of the approximately cylindrical sidewall to a container. A floating ball valve may be disposed within the upper portion being constructed and arranged to function as a one-way valve such that escape gases and fluids may escape the container while also preventing environmental fluids and contaminants from passing through the flexible lid into a container.

### TECHNICAL FIELD

The embodiments generally relate to removable lids for containers.

### BACKGROUND

Fermentation vessels and containers may utilize airlocks to allow fluids, such as gases, formed during fermentation to escape the container while preventing undesirable fluids and contaminants from entering the container through the airlock. Containers may utilize airlocks airlocks to create an airtight seal such that goods, such as dry goods, are not exposed to atmospheric conditions. Airlocks may utilize a generally S-shaped channel in which a fluid may be stored. The fluid may allow fermentation gases to escape the container through the airlock while preventing undesirable fluids and contaminants from entering the container. However, where fermentation is highly active, large volumes of escape gases may unintentionally force the airlock fluid out of the airlock thereby rendering the airlock ineffective and potentially causing contamination of the container and products therein.

### SUMMARY

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

A flexible lid may include an elastic body including an upper portion connected approximately perpendicular to an approximately cylindrical sidewall, the elastic body defining a recess and inner wall constructed and arranged to facilitate the attachment of the flexible lid to a container. The flexible lid may be constructed and arranged to be attachable to a variety of containers such as, but not limited to, glass or plastic cups, mason jars, or other jars or containers having smooth lips or threaded lips meant for mating with more traditional threaded lids.

The flexible lid may further include a plurality of circumferential ribs integrally formed with the approximately cylindrical sidewall constructed and arranged to facilitate the elastic strength of the approximately cylindrical sidewall such as the flexible lid may be tightly secured to a container. A flange extending radially from the approximately cylindrical sidewall may be constructed and arranged to facilitate ease of attachment of the flexible lid to a container such that a user may grip the flange during use. The flange may include at least one structural support ridge integrally formed with the flange and constructed and arranged to support the flange.

The flexible lid may further include a floating ball valve integrally formed with the upper portion including a vent plate defining a vent plate hole, the upper portion include a lower lip, and a ball disposed between the vent plate and the lower lip, the floating ball valve being constructed and arranged to function as a one-way valve in the elastic body.

The flexible lid may create an airtight seal with a container while also providing for one-way valve functionality in that fluids such as escape gases may escape the container through the floating ball valve while simultaneously preventing environment contaminants from entering the container. For example, the flexible lid may allow the escape of gases from a container containing fermentable materials which may create fluids such as carbon dioxide during fermentation.

The flexible lid may be elastically stretched around the mouth of a container such as a jar to create an airtight seal within the container. For example, the flexible lid may be used to create an airtight seal for containers of dry goods such as coffee, beans, spices, or the like to prevent the external atmosphere from reaching the goods within the container.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a perspective view of a flexible lid according to some variations described herein;

FIG. 2 illustrates a top view of a flexible lid according to some variations described herein;

FIG. 3 illustrates a cross-sectional view of a flexible lid according to some variations described herein;

FIGS. 4a and 4b illustrate a view of a floating ball valve within a flexible lid according to some variations described herein;

FIG. 5 illustrates a perspective view of a flexible lid according to some variations described herein; and

FIG. 6 illustrates a perspective view of a flexible lid on a container according to some variations described herein;

### DETAILED DESCRIPTION

The specific details of the single embodiment or variety of embodiments described herein are to the described system and methods of use. Any specific details of the embodiments are used for demonstration purposes only and no unnecessary limitations or inferences are to be understood from there.

It is noted that the embodiments reside primarily in combinations of components and procedures related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

The scope of the invention will be best understood by the following description of figures.

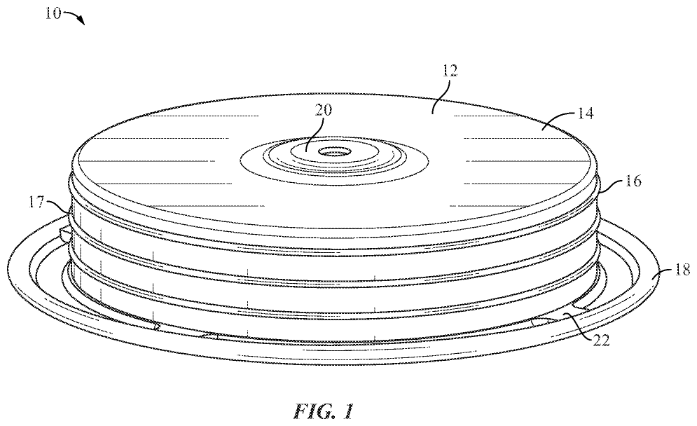

FIG. 1 depicts a variation of a flexible lid 10 including a body 12 incorporating a ball valve 20 therein. The body 12 may include an upper portion having an upper surface 14 connected approximately perpendicular to a cylindrical sidewall 16 extending radially into a flange 18. Sidewall 16 may include a plurality of ribs 17 constructed and arranged to provide structural support to the sidewall 16. The plurality of ribs 17 may be further constructed and arranged to facilitate the elastic seal of the sidewall 16 against a container or vessel on to which the flexible lid 10 may be secured. Flange 18 may include structural support ridges 22.

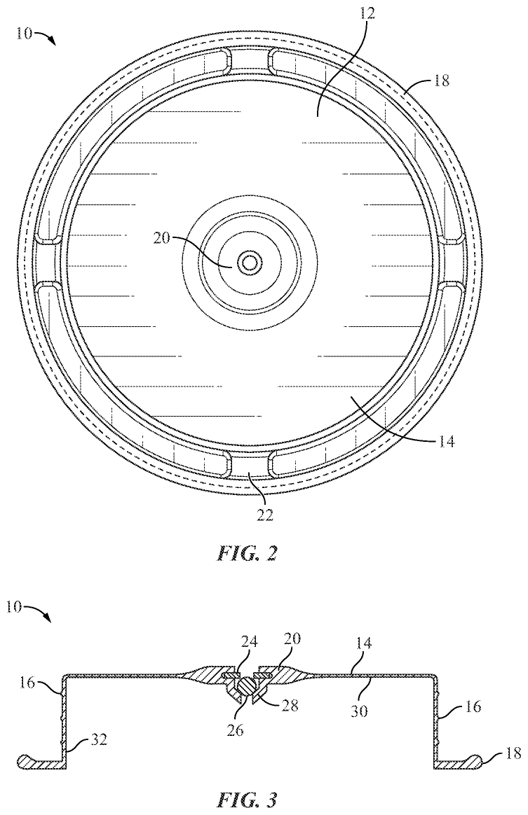

FIG. 2 depicts a variation of a flexible lid 10 including a body 12 having an upper surface 14 which may include a decorative element or embossment thereon. The ball valve 20 may be integrated into the body 12 at about the center point of the upper surface 14. Flange 18 and structural support ridge 22 may extend from the sidewall 16.

FIG. 3 depicts a cross-sectional view of a variation of a flexible lid 10 including a body having an upper surface 14 and a ball valve incorporated into the body. The sidewall 16 may extend from the upper surface 14 and may be connected to the flange 18. The body may define a recess 34 and an inner wall 32 constructed and arranged to facilitate the attachment of the flexible lid 10 to a container, such as a glass jar.

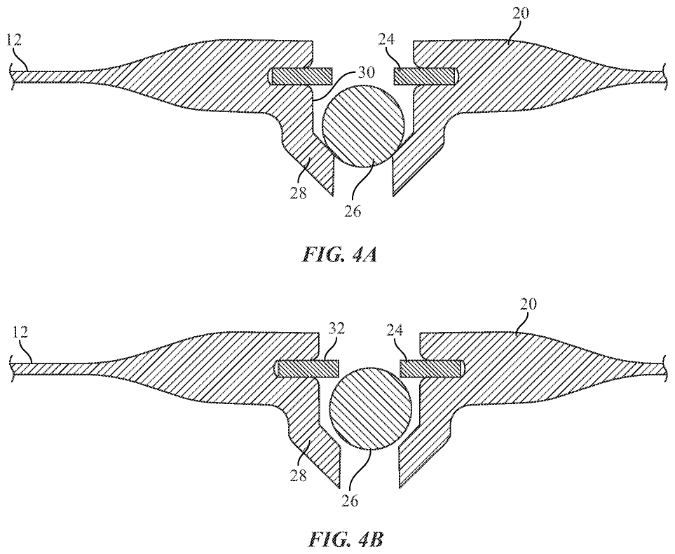

FIGS. 4a and 4b depict a cross-sectional view of a floating ball valve within a flexible lid 10. The ball valve 20 may define a vent hole 30 in the flexible lid 10 through which gas or fluid may pass. The ball valve 20 may include a lower lip 28 constructed and arranged to support a ball 26 seated thereon such that fluid may not enter a vessel onto which the flexible lid 10 may be attached during use. The ball valve 20 may include a vent plate 24 defining a vent plate hole 32 constructed and arranged to prevent the ball 26 from escaping the vent hole 30 in circumstances where fluid may be flowing through the vent hole 30 and out of a container.

FIG. 5 depicts a variation of a flexible lid including a body 12 having an upper surface 14 which may include a decorative element or embossment thereon. The ball valve 20 may be integrated into the body 12 at about the center point of the upper surface 14. The ball valve 20 may include a valve cover 40 defining valve cover perforations 42 constructed and arranged to further prevent the ball 26 from escaping the vent hole 30 in circumstances where fluid may be flowing through the vent hole 30 and out of a container. Flange 18 and structural support ridge 22 may extend from the sidewall.

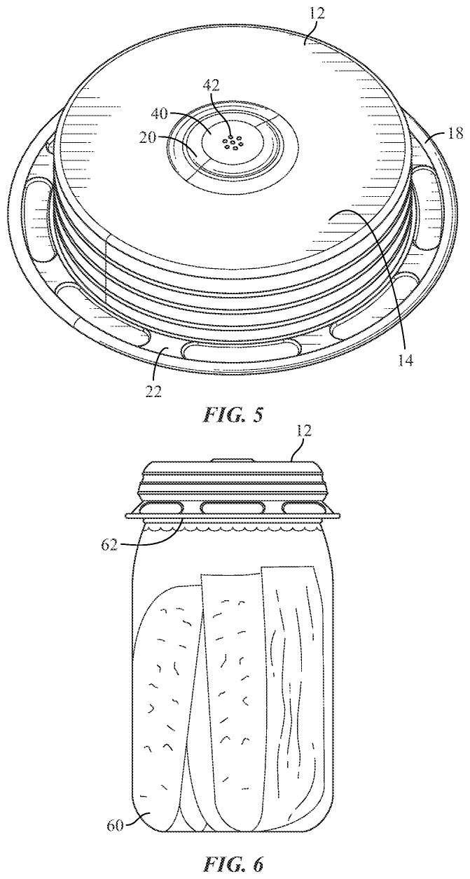

FIG. 6 depicts a variation of a flexible lid including a flexible body 12 disposed over an opening 62 of a container 60. The container 60 may be any container having an opening such as, but not limited to, cans, jars, cups, bowls, glass or plastic food containers, or the like.

In use, the flexible lid may be elastically stretched around the mouth of a container such as a glass jar to prevent fluids from entering the container. The ball may be seated within the ball valve naturally due to gravity such that fluid may not enter the container. The container may be used for methods such as fermentation which may produce fluid in the form of escape gases and the flexible lid may be constructed and arranged to allow gas to escape from the container through the ball valve by floating the ball temporarily where internal pressure of the container exceeds that of atmospheric pressure such that gases may escape the container momentarily until container pressure is at or about the same as atmospheric pressure and the ball may descend and be held by the lower lip of the ball valve thereby preventing atmospheric fluids from entering the container. The vent plate may prevent the ball from a scaping t-ball valve.

The flexible lid may be made of any material exhibiting flexible or elastic properties such as rubber, plastic, or the like. The flexible lid may be constructed and arranged to a elastically seal a variety of containers and may come in a variety of sizes such that appropriately sized flexible lids may be used on corresponding vessels and containers.

The vent plate may be generally disc shaped and may define event plate hole therein. The vent plate may be seated within a generally disc shaped recess defined by the ball valve. In some variations, the ball valve may be made of the same elastic material as the flexible lid such that the vent plate may be removable from the ball valve, changeable, or customizable with respect to desired one-way ball valve operation.

The following description of variants is only illustrative of components, elements, acts, products, and methods considered to be within the scope of the invention and are not in any way intended to limit such scope by what is specifically disclosed or not expressly set forth. The components, elements, acts, products, and methods as described herein may be combined and rearranged other than as expressly described herein and are still considered to be within the scope of the invention.

According to variation 1, a flexible lid may include a body including an upper portion connected approximately perpendicular to an approximately cylindrical sidewall, the body defining a recess and inner wall constructed and arranged to facilitate the attachment of the flexible lid to a container. The flexible lid may include a plurality of ribs integrally formed with the approximately cylindrical sidewall constructed and arranged to facilitate the elastic compression of the approximately cylindrical sidewall and a floating ball valve disposed within the upper portion being constructed and arranged to function as a one-way valve.

Variation 2 may include a flexible lid as in variation 1 wherein the floating ball valve may be integrally formed with the upper portion.

Variation 3 may include a flexible lid as in any of variations 1 through 2 further including a vent plate defining a vent plate hole therein, the vent plate being seated within a portion of the vent hole.

Variation 4 may include a flexible lid as in any of variations 1 through 3 wherein the vent plate may be integrally formed with the floating ball valve.

Variation 5 may include a flexible lid as in any of variations 1 through 4 wherein the floating ball valve defines a vent hole in the flexible lid and includes a lower lip constructed and arranged to support a ball seated thereon and further including a vent plate defining a vent plate hole therein, the vent plate being seated within a portion of the vent hole.

Variation 6 may include a flexible lid as in any of variations 1 through 5 wherein the vent plate may be constructed and arranged to prevent the ball from being removed from the vent hole.

Variation 7 may include a flexible lid as in any of variations 1 through 6 wherein the lower lip may be constructed and arranged to prevent the ball from being removed from the vent hole.

Variation 8 may include a flexible lid as in any of variations 1 through 7 wherein the vent hole may be constructed and arranged to allow the ball to float therein between the lower lip in the vent plate.

Variation 9 may include a flexible lid as in any of variations 1 through 8 wherein the body includes a material exhibiting flexible or elastic properties.

Variation 10 may include a flexible lid as in any of variations 1 through 9 further including a flange extending radially from the approximately cylindrical sidewall and being constructed and arranged to facilitate ease of attachment of the flexible lid to a container.

According to variation 11, a flexible lid may include a body including an upper portion connected approximately perpendicular to an approximately cylindrical sidewall, the body defining a recess and inner wall constructed and arranged to facilitate the attachment of the flexible lid to a container; a plurality of ribs integrally formed with the approximately cylindrical sidewall constructed and arranged to facilitate the elastic compression of the approximately cylindrical sidewall; and a flange extending radially from the approximately cylindrical sidewall and being constructed and arranged to facilitate ease of attachment of the flexible lid to a container. The flexible lid may further include a floating ball valve integrally formed with the upper portion being constructed and arranged to function as a one-way valve wherein the floating ball valve defines a vent hole in the flexible lid and includes a lower lip constructed and arranged to support a ball seated thereon and further including a vent plate defining a vent plate hole therein, the vent plate being seated within a portion of the vent hole.

Variation 12 may include a flexible lid as in variation 11 wherein the vent plate may be integrally formed with the floating ball valve.

Variation 13 may include a flexible lid as in any of variations 11 through 12 wherein the vent plate may be constructed and arranged to prevent the ball from being removed from the vent hole.

Variation 14 may include a flexible lid as in any of variations 11 through 13 wherein the lower lip may be constructed and arranged to prevent the ball from being removed from the vent hole.

Variation 15 may include a flexible lid as in any of variations 11 through 14 wherein the vent hole may be constructed and arranged to allow the ball to float therein between the lower lip in the vent plate.

Variation 16 may include a flexible lid as in any of variations 11 through 15 wherein the body includes a material exhibiting flexible or elastic properties.

Variation 17 may include a flexible lid as in any of variations 11 through 16 wherein the flange includes a plurality of structural support ridges.

According to variation 18, a flexible lid may include an elastic body including an upper portion connected approximately perpendicular to an approximately cylindrical sidewall, the elastic body defining a recess and inner wall constructed and arranged to facilitate the attachment of the flexible lid to a container. The flexible lid may further include a plurality of circumferential ribs integrally formed with the approximately cylindrical sidewall constructed and arranged to facilitate the elastic strength of the approximately cylindrical sidewall; a flange extending radially from the approximately cylindrical sidewall and being constructed and arranged to facilitate ease of attachment of the flexible lid to a container; and at least one structural support ridge integrally formed with the flange and constructed and arranged to support the flange. The flexible lid may further include a floating ball valve integrally formed with the upper portion including a vent plate defining a vent plate hole, a lower lip, and a ball disposed between the vent plate and the lower lip, the floating ball valve being constructed and arranged to function as a one-way valve in the elastic body.

Variation 19 may include a flexible lid as in variation 18 wherein the vent plate may be integrally formed with the floating ball valve.

Variation 20 may include a flexible lid as in any of variations 18 through 19 wherein the vent hole may be constructed and arranged to allow the ball to float therein between the lower lip in the vent plate.

Many different embodiments have been disclosed herein, in connection with the above description and the drawing. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for anyone of the elements in the claims below or that a single element can be substituted for two or more elements in a claim. Although elements can be described above as acting in certain combinations, and even initially claimed as such, it is to be expressly understood that one or more elements from a claimed combination can, in some cases, be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible considering the above teachings without departing from the following claims.

What is claimed is:

1. A flexible lid comprising:
a body comprising an upper portion connected approximately perpendicular to an approximately cylindrical sidewall, the body defining a recess and inner wall constructed and arranged to facilitate the attachment of the flexible lid to a container;
a plurality of ribs integrally formed with the approximately cylindrical sidewall constructed and arranged to facilitate the elastic compression of the approximately cylindrical sidewall; and
a floating ball valve disposed within the upper portion being constructed and arranged to function as a one-way valve.

2. A flexible lid as in claim 1 wherein the floating ball valve is integrally formed with the upper portion.

3. A flexible lid as in claim 1 further comprising a vent plate defining a vent plate hole therein, the vent plate being seated within a portion of the vent hole.

4. A flexible lid as in claim 1 wherein the vent plate is integrally formed with the floating ball valve.

5. A flexible lid as in claim 1 wherein the floating ball valve defines a vent hole in the flexible lid and comprises a lower lip constructed and arranged to support a ball seated thereon and further comprising a vent plate defining a vent plate hole therein, the vent plate being seated within a portion of the vent hole.

6. A flexible lid as in claim 1 wherein the vent plate is constructed and arranged to prevent the ball from being removed from the vent hole.

7. A flexible lid as in claim 1 wherein the lower lip is constructed and arranged to prevent the ball from being removed from the vent hole.

8. A flexible lid as in claim 1 wherein the vent hole is constructed and arranged to allow the ball to float therein between the lower lip in the vent plate.

9. A flexible lid as in claim 1 wherein the body comprises a material exhibiting flexible or elastic properties.

10. A flexible lid as in claim 1 further comprising a flange extending radially from the approximately cylindrical sidewall and being constructed and arranged to facilitate ease of attachment of the flexible lid to a container.

11. A flexible lid comprising:
a body comprising an upper portion connected approximately perpendicular to an approximately cylindrical sidewall, the body defining a recess and inner wall constructed and arranged to facilitate the attachment of the flexible lid to a container;
a plurality of ribs integrally formed with the approximately cylindrical sidewall constructed and arranged to facilitate the elastic compression of the approximately cylindrical sidewall;
a flange extending radially from the approximately cylindrical sidewall and being constructed and arranged to facilitate ease of attachment of the flexible lid to a container; and
a floating ball valve integrally formed with the upper portion being constructed and arranged to function as a one-way valve wherein the floating ball valve defines a vent hole in the flexible lid and comprises a lower lip constructed and arranged to support a ball seated thereon and further comprising a vent plate defining a vent plate hole therein, the vent plate being seated within a portion of the vent hole.

12. A flexible lid as in claim 1 wherein the vent plate is integrally formed with the floating ball valve.

13. A flexible lid as in claim 1 wherein the vent plate is constructed and arranged to prevent the ball from being removed from the vent hole.

14. A flexible lid as in claim 1 wherein the lower lip is constructed and arranged to prevent the ball from being removed from the vent hole.

15. A flexible lid as in claim 1 wherein the vent hole is constructed and arranged to allow the ball to float therein between the lower lip in the vent plate.

16. A flexible lid as in claim 1 wherein the body comprises a material exhibiting flexible or elastic properties.

17. A flexible lid as in claim 1 wherein the flange comprises a plurality of structural support ridges.

18. A flexible lid comprising:
an elastic body comprising an upper portion connected approximately perpendicular to an approximately cylindrical sidewall, the elastic body defining a recess and inner wall constructed and arranged to facilitate the attachment of the flexible lid to a container;
a plurality of circumferential ribs integrally formed with the approximately cylindrical sidewall constructed and arranged to facilitate the elastic strength of the approximately cylindrical sidewall;
a flange extending radially from the approximately cylindrical sidewall and being constructed and arranged to facilitate ease of attachment of the flexible lid to a container;
at least one structural support ridge integrally formed with the flange and constructed and arranged to support the flange; and
a floating ball valve integrally formed with the upper portion comprising a vent plate defining a vent plate hole, a lower lip, and a ball disposed between the vent plate and the lower lip, the floating ball valve being constructed and arranged to function as a one-way valve in the elastic body.

19. A flexible lid as in claim 1 wherein the vent plate is integrally formed with the floating ball valve.

20. A flexible lid as in claim 1 wherein the vent hole is constructed and arranged to allow the ball to float therein between the lower lip in the vent plate.
